(19) United States
(12) Patent Application Publication
(10) Pub. No.: US 2024/0065884 A1
(43) Pub. Date: Feb. 29, 2024

(54) Pad Designs to Improve Delivery of Temperature Therapy

(71) Applicant: C. R. Bard, Inc., Franklin Lakes, NJ (US)

(72) Inventors: Eric A. Fallows, Apex, NC (US); Nicholas J. Jardine, Holly Springs, NC (US); Qihua Xu, Cary, NC (US); Adam T. Martin, Holly Springs, NC (US); Sean E. Walker, Platteville, CO (US)

(21) Appl. No.: 18/272,026

(22) PCT Filed: Jan. 11, 2022
(86) PCT No.: PCT/US22/11980

§ 371 Date: Jul. 12, 2023

Related U.S. Application Data
(60) Provisional application No. 63/136,335, filed on Jan. 12, 2021.

(51) Int. Cl. A61F 7/02; A61F 7/00

(52) U.S. Cl. CPC A61F 7/02; A61F 7/0085; A61F 2007/0054

(57) ABSTRACT

Disclosed herein are systems and methods for providing targeted temperature management (TTM) therapy to a patient. The TTM system can include multiple embodiments of a thermal pad including embodiments that are convertible from a first patient contact area to a second patient contact area. Embodiments of thermal pads can include pads that are expandable, extendable, and/or comprise attachable or removable portions. The TTM system can include embodiments where a second thermal pad is coupled to a first thermal pad.

### PRIORITY

This application claims the benefit of priority to U.S. Provisional Application No. 63/136,335, filed Jan. 12, 2021, which is incorporated by reference in its entirety into this application.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g. water) through one or more thermal contact pads coupled to a patient to affect surface-to-surface thermal energy exchange with the patient. In general, TTM systems comprise a TTM fluid control module coupled to at least one contact pad via a fluid deliver line. One such TTM system is disclosed in U.S. Pat. No. 6,645,232, titled “Patient Temperature Control System with Fluid Pressure Maintenance” filed Oct. 11, 2001 and one such thermal contact pad and related system is disclosed in U.S. Pat. No. 6,197,045 titled “Cooling/heating Pad and System” filed Jan. 4, 1999, both of which are incorporated herein by reference in their entireties. As noted in the '045 patent, the ability to establish and maintain intimate pad-to-patient contact is of importance to fully realizing medical efficacies with TTM systems.

As these and other medical applications have evolved, the accommodation of different patient sizes has become more important. Disclosed herein are embodiments of devices and methods for the adjusting the patient contact area of the thermal pad to better accommodate patients of different sizes.

### SUMMARY OF THE INVENTION

Briefly summarized, disclosed herein is a medical pad for exchanging thermal energy between a targeted temperature management (TTM) fluid and a patient. The pad may comprise a fluid containing layer for containing the TTM fluid, where the fluid containing layer comprises a fluid inlet and a fluid outlet and where the TTM fluid is circulatable within the fluid containing layer from the fluid inlet to the fluid outlet. The pad may further comprise a patient contact surface defining a patient contact area to facilitate thermal energy exchange with the patient and the patient contact area may be convertible from a first patient contact area to a second patient contact area, where the second patient contact area is different from the first patient contact area.

In some embodiments, the second patient contact area is at least 10 percent greater than the first patient contact area. The patient contact area may be expandable from the first patient contact area to the second patient contact area and in some embodiments, the patient contact area is expandable along a single dimension. The pad may comprise one or more folds to facilitate expansion from the first patient contact area to the second patient contact area patient and in some embodiments, the pad comprises a stretchable material to facilitate expansion from the first patient contact area to the second patient contact area.

In some embodiments, the pad may further comprise a first pad defining the first patient contact area and a second pad connectable to the first pad and the second pad includes a separate second patient contact area, and converting the first patient contact area to the second patient contact area may comprise connecting the second pad to the first pad thereby combining the first patient contact area with the separate second patient contact area to the define the second patient contact area. The first pad includes a first fluid containing layer, the second pad includes a second fluid containing layer, and connecting the second pad to the first pad may include fluidly coupling the second fluid containing layer to the fluid containing layer so that the TTM fluid is circulatable within the second fluid containing layer.

In some embodiments, converting the first patient contact area to the second patient contact area comprises separating a removable portion of the pad from a remaining portion of the pad. The removable portion may be disposed along an entire circumference of the remaining portion or a partial perimeter of the remaining portion. The removable portion comprises a removable fluid containing layer, the remaining portion comprises a remaining fluid containing layer, and the pad comprises one or more fluid conduits extending between the remaining fluid containing layer and the removable fluid containing layer. In some embodiments, converting the first patient contact area to the second patient contact area comprises separating a removable portion of the one or more conduits from a remaining portion of the one or more conduits and occluding the remaining portion of the one or more fluid conduits.

In some embodiments, the pad is configured to be disposed on a bed surface, and the first contact area is defined by a portion of the pad disposed between the patient and the bed surface. The pad may comprise one or more lateral extensions extending away from the patient along the bed surface, and converting the first contact area to the second contact area comprises wrapping at least a portion of the one or more lateral extensions around a portion of the patient to define the second contact area. The portion of the patient may comprise at least a portion of a torso of the patient. The pad may comprise one or more straps to secure the one or more lateral extensions around the portion of the patient.

In some embodiments, The thermal pad comprises a first portion of the pad including a first portion of the patient contact area is separable from a second portion of the pad comprising a second portion of the patient contact area, and wherein the first portion of the pad is coupled to the second portion of the pad via an extendable member. The extendable member comprises one or more fluid conduits extending between the first portion of the pad and the second portion of the pad.

In some embodiments, the fluid containing layer comprises a TTM fluid flow path, and the pad further comprises a filter disposed in line with the TTM fluid flow path.

Disclosed herein is also a method of providing a targeted temperature management (TTM) therapy to a patient. The method comprises providing a TTM system comprising that includes a TTM module configured to provide a TTM fluid, a thermal pad configured to receive the TTM fluid from the TTM module to facilitate thermal energy transfer between the TTM fluid and a patient, and a fluid delivery line (FDL) extending between the TTM module and the FDL is configured to provide TTM fluid flow between the TTM module and the thermal pad. The thermal pad comprises a patient contact surface defining a patient contact area to facilitate thermal energy exchange with the patient and the patient contact area is convertible from a first patient contact area to a second patient contact area, wherein the second patient contact area is different from the first patient contact area. The method further includes applying the pad to the patient and delivering TTM fluid from the TTM module to the thermal pad.

The method may comprise expanding the pad along a single dimension to convert the first patient contact area to the second patient contact area. The method may comprise stretching a material of the pad to convert the first patient contact area to the second patient contact area. The method may comprise coupling a second thermal pad to the first thermal pad, where the first thermal pad defines the first patient contact area, and the first patient contact area is combined with a separate second patient contact area of the second thermal pad to define the second patient contact area. The method may comprise separating a removable portion of the thermal pad from a remaining portion of the thermal pad to convert the first patient contact area to the second patient contact area.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a targeted temperature management (TTM) system for cooling or warming a patient, in accordance with some embodiments.

FIG. 2 illustrates a hydraulic schematic of the TTM system of FIG. 1, in accordance with some embodiments.

FIG. 3 illustrates a block diagram depicting various elements of a console of the TTM module of FIG. 1, in accordance with some embodiments.

FIG. 4A is a top view of the portion the thermal contact pad of FIG. 1, in accordance with some embodiments.

FIG. 4B is a cross-sectional side view of the portion the thermal contact pad of FIG. 4A, in accordance with some embodiments.

FIG. 5A is an illustration of an expandable thermal pad, in accordance with some embodiments.

FIG. 5B is a cross-sectional view of a portion of the expandable thermal pad of FIG. 5A, in accordance with some embodiments.

FIG. 6A is an illustration of a thermal pad that may be disposed between a patient and a surface of a bed, in accordance with some embodiments.

FIG. 6B is an illustration of a thermal pad of FIG. 6A wrapped around a torso of a patient, in accordance with some embodiments.

FIG. 7A is an illustration of a thermal pad comprising a removable portion, in accordance with some embodiments.

FIG. 7B is detail view of a portion thermal pad of FIG. 7A, in accordance with some embodiments.

FIG. 7C is detail view of a remaining portion of the thermal pad of FIG. 7A, in accordance with some embodiments.

FIG. 8A is an illustration of thermal pad comprising an extendable portion in a non-extended state, in accordance with some embodiments.

FIG. 8B is an illustration of the thermal pad of FIG. 8A with the extendable portion in an extended state, in accordance with some embodiments.

FIG. 9A is an illustration of a thermal pad system comprising a first pad fluidly connectable to a second pad, in accordance with some embodiments.

FIG. 9B is an illustration of a connector set of the thermal pad system of FIG. 9A, where the connector set is disposed in a disconnected state, in accordance with some embodiments.

FIG. 9C is an illustration of the connector set of FIG. 9B disposed in a connected state, in accordance with some embodiments.

FIG. 10A provides an exploded perspective view of a TTM fluid filter, in accordance with some embodiments.

FIG. 10B provides a cross-sectional side view of the filter of FIG. 10A, in accordance with some embodiments.

FIG. 10C provides a side cross-sectional view of the thermal contact pad of FIG. 1 incorporating the filter of FIG. 10A, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled to” refer to any form of interaction between two or more entities, including mechanical, electrical, magnetic, electromagnetic, fluid, signal, communicative (including wireless), and thermal interaction. Two components may be connected or coupled to each other even though they are not in direct contact with each other. For example, two components may be coupled to each other through an intermediate component.

Unless defined otherwise, all technical and scientific terms used herein have the same meaning as commonly understood by those of ordinary skill in the art.

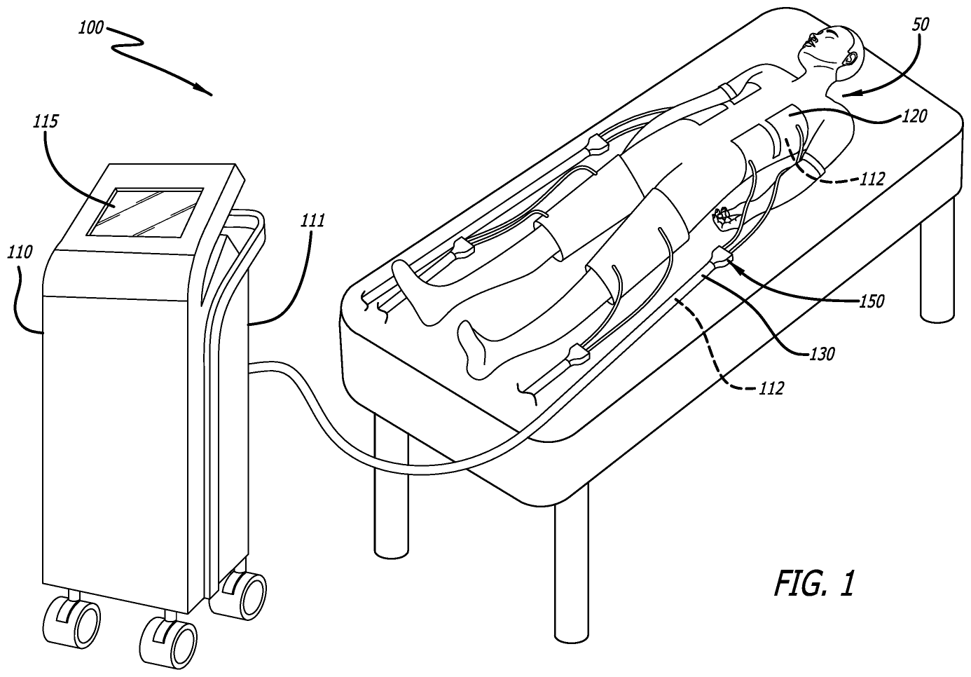

FIG. 1 illustrates a targeted temperature management (TTM) system 100 connected to a patient 50 for administering targeted temperature management therapy to the patient 50 which may include a cooling and/or warming of the patient 50, in accordance with some embodiments. The TTM system 100 comprises a TTM module 110 including a graphical user interface (GUI) 115 enclosed within a module housing 111. The TTM system 100 includes a fluid deliver line (FDL) 130 extending from the TTM module 110 to a thermal contact pad assembly 120 to provide for flow of TTM fluid 112 between the TTM module 110 and the pad assembly 120. The FDL includes two conduits to facilitate delivery flow of TTM fluid 112 from the TTM module 110 to the pad assembly 120 and return flow TTM fluid 112 from the pad assembly 120 to the TTM module 110. In some embodiments, the two conduits may be attached to each other along a portion of a length of the FDL.

The TTM system 100 may include 1, 2, 3, 4 or more pads 120 and the TTM system 100 may include 1, 2, 3, 4 or more fluid delivery lines 130. In use, the TTM module 110 prepares the TTM fluid 112 for delivery to the pad assembly 120 by heating or cooling the TTM fluid 112 to a defined temperature in accordance with a prescribed TTM therapy. The TTM module 110 circulates the TTM fluid 112 along a TTM fluid flow path including within the pad assembly 120 to facilitate thermal energy exchange with the patient 50. During the TTM therapy, the TTM module 110 may continually control the temperature of the TTM fluid 112 toward a target TTM temperature.

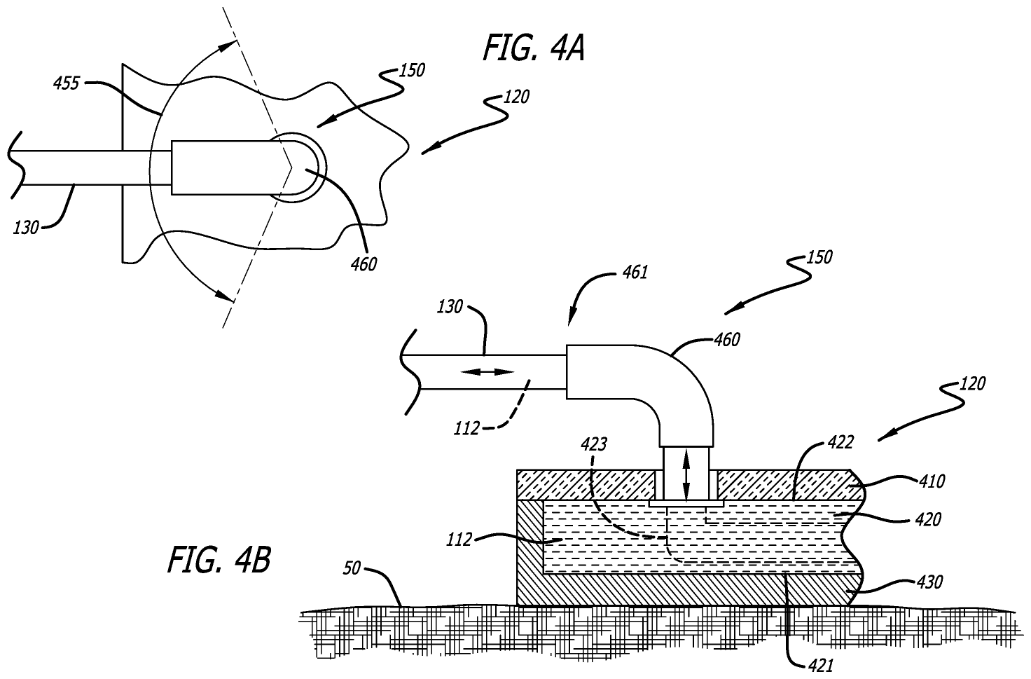

The TTM system 100 may include a connector system 150 to couple the FDL 130 to the pad assembly 120. In some embodiments, the connector system 150 may couple a single fluid conduit of the FDL to the pad assembly 120. Hence, the connection between the FDL 130 and the pad assembly 120 may comprise more than one connector system 150 to couple more than one fluid conduit to the pad assembly 120. The connector system 150 is further described below in FIGS. 4A and 4B.

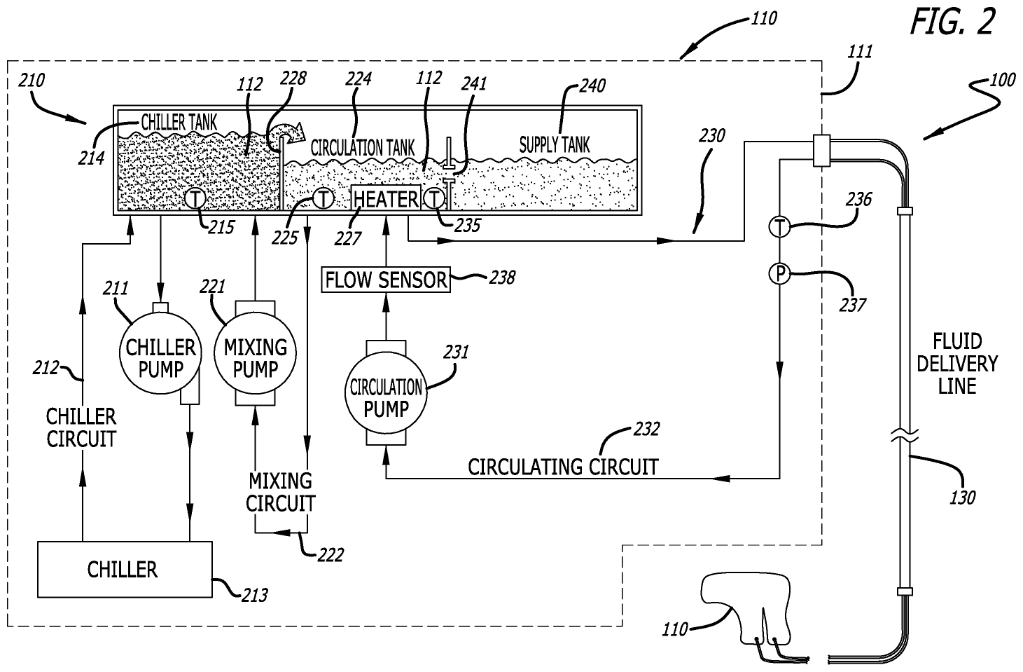

FIG. 2 illustrates a hydraulic schematic of the TTM system 100. The FDL 130 and the pad assembly 120 are disposed external to the housing 111 of the TTM module 110. The TTM module includes various fluid sensors and fluid control devices to prepare and circulate the TTM fluid 112. The fluid subsystems of the TTM module may include a temperature control subsystem 210 and a circulation subsystem 230.

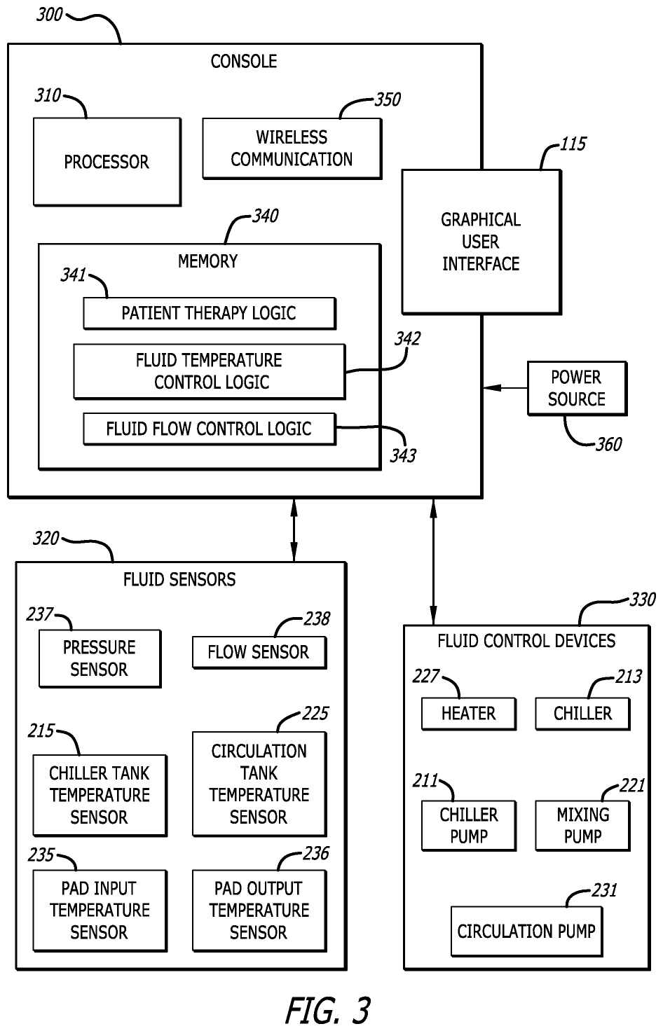

The temperature control subsystem 210 may include a chiller pump 211 to pump (recirculate) TTM fluid 112 through a chiller circuit 212 that includes a chiller 213 and a chiller tank 214. A temperature sensor 215 within the chiller tank 214 is configured to measure a temperature of the TTM fluid 112 within the chiller tank 214. The chiller 213 may be controlled by a temperature control logic (see FIG. 3) as further described below to establish a desired temperature of the TTM fluid 112 within chiller tank 214. In some instances, the temperature of the TTM fluid 112 within the chiller tank 214 may be less than the target temperature for the TTM therapy.

The temperature control subsystem 210 may further include a mixing pump 221 to pump TTM fluid 112 through a mixing circuit 222 that includes the chiller tank 214, a circulation tank 224, and a dam 228 disposed between the chiller tank 214 and circulation tank 224. The TTM fluid 112, when pumped by the mixing pump 221, enters the chiller tank 214 and mixes with the TTM fluid 112 within the chiller tank 214. The mixed TTM fluid 112 within the chiller tank 214 flows over the dam 228 and into the circulation tank 224. In other words, the mixing circuit 222 mixes the TTM fluid 112 within chiller tank 214 with the TTM fluid 112 within circulation tank 224 to cool the TTM fluid 112 within the circulation tank 224. A temperature sensor 225 within the circulation tank 224 measures the temperature of the TTM fluid 112 within the circulation tank 224. The temperature control logic may control the mixing pump 221 in accordance with temperature data from the temperature sensor 225 within the circulation tank 224.

The circulation tank 224 includes a heater 227 to increase to the temperature of the TTM fluid 112 within the circulation tank 224, and the heater 227 may be controlled by the temperature control logic. In summary, the temperature control logic when executed by the processor (see FIG. 3) may 1) receive temperature data from the temperature sensor 215 within the chiller tank and the temperature sensor 225 within the circulation tank 224 and 2) control the operation of the chiller 213, the chiller pump 211, the heater 227, and mixing pump 222 to establish and maintain the temperature of the TTM fluid 112 within the circulation tank 224 at the target temperature for the TTM therapy.

The circulation subsystem 230 comprises a circulation pump 213 to pull TTM fluid 112 from the circulation tank 224 and through a circulating circuit 232 that includes the fluid delivery line 130 and the pad assembly 120 located upstream of the circulation pump 213. The circulating circuit 232 also includes a pressure sensor 237 to represent a pressure of the TTM fluid 112 within the pad assembly 120. The circulating circuit 232 includes a temperature sensor 235 within the circulation tank 224 to represent the temperature of the TTM fluid 112 entering the pad assembly 120 and a temperature sensor 236 to represent the temperature of the TTM fluid exiting the pad assembly 120. A flow meter 238 is disposed downstream of the circulation pump 213 to measure the flow rate of TTM fluid 112 through the circulating circuit 232 before the TTM fluid 112 re-enters that the circulation tank 224.

In use, the circulation tank 224, which may be vented to atmosphere, is located below (i.e., at a lower elevation than) the pad assembly 120 so that a pressure within the pad assembly 120 is less than atmospheric pressure (i.e., negative) when TTM fluid flow through the circulating circuit 232 is stopped. The pad assembly 120 is also placed upstream of the circulation pump 231 to further establish a negative pressure within the pad assembly 120 when the circulation pump 213 is operating. The fluid flow control logic (see FIG. 3) may control the operation of the circulation pump 213 to establish and maintain a desired negative pressure within the pad assembly 120. A supply tank 240 provides TTM fluid 112 to the circulation tank 224 via a port 241 to maintain a defined volume of TTM fluid 112 within the circulation tank 224.

FIG. 3 illustrates a block diagram depicting various elements of the TTM module 110 of FIG. 1, in accordance with some embodiments. The TTM module 110 includes a console 300 including a processor 310 and memory 340 including non-transitory, computer-readable medium. Logic modules stored in the memory 340 include patient therapy logic 341, fluid temperature control logic 342, and fluid flow control logic 343. The logic modules when executed by the processor 310 define the operations and functionality of the TTM Module 110.

Illustrated in the block diagram of FIG. 3 are fluid sensors 320 as described above in relation to FIG. 2. Each of the fluid sensors 320 are coupled to the console 300 so that data from the fluid sensors 320 may be utilized in the performance of TTM module operations. Fluid control devices 330 are also illustrated in FIG. 3 as coupled to the console 300. As such, logic modules may control the operation of the fluid control devices 330 as further described below.

The patient therapy logic 341 may receive input from the clinician via the GUI 115 to establish operating parameters in accordance with a prescribed TTM therapy. Operating parameters may include a target temperature for the TTM fluid 112 and/or a thermal energy exchange rate which may comprise a time-based target temperature profile. In some embodiments, the fluid temperature control logic 342 may define other fluid temperatures of the TTM fluid 112 within the TTM module 110, such a target temperature for the TTM fluid 112 within the chiller tank 214, for example.

The fluid temperature control logic 342 may perform operations to establish and maintain a temperature of the TTM fluid 112 delivered to the pad assembly 120 in accordance with the predefined target temperature. One temperature control operation may include chilling the TTM fluid 112 within the chiller tank 214. The fluid temperature control logic 342 may utilize temperature data from the chiller tank temperature sensor 215 to control the operation of the chiller 213 to establish and maintain a temperature of the TTM fluid 112 within the chiller tank 214.

Another temperature control operation may include cooling the TTM fluid 112 within the circulation tank 224. The fluid temperature control logic 342 may utilize temperature data from the circulation tank temperature sensor 225 to control the operation of the mixing pump 221 to decrease the temperature of the TTM fluid 112 within the circulation tank 224 by mixing TTM fluid 112 from the chiller tank 214 with TTM fluid 112 within circulation tank 224.

Still another temperature control operation may include warming the TTM fluid 112 within the circulation tank 224. The fluid temperature control logic 342 may utilize temperature data from the circulation tank temperature sensor 225 to control the operation of the heater 227 to increase the temperature of the TTM fluid 112 within the circulation tank 224.

The fluid flow control logic 343 may control the operation of the circulation pump 231. As a thermal energy exchange rate is at least partially defined by the flow rate of the TTM fluid 112 through the pad assembly 120, the fluid flow control logic 343 may, in some embodiments, control the operation of the circulation pump 231 in accordance with a defined thermal energy exchange rate for the TTM therapy.

The console 300 may include or be couple do wireless communication module 350 to facilitate wireless communication with external devices. A power source 360 provides electrical power to the console 300.

FIG. 4A shows a top view of a portion of the thermal contact pad assembly 120 including the connector system 150 and the FDL 130 extending away from the connector system 150, in accordance with some embodiments. As illustrated, the connector system 150 may provide for a rotatable connection between the FDL 130 and the pad assembly 120. The rotatable connection may provide for the FDL 130 to rotate over an angle 455 ranging up to about 90 degrees, 180 degrees, or 360 degrees.

FIG. 4B shows a cross-sectional side view of a portion of the thermal contact pad assembly 120 in contact with the patient 50, in accordance with some embodiments. The pad assembly 120 may comprise multiple layers to provide multiple functions of the pad assembly 120. A fluid containing layer 420 is fluidly coupled to the FDL 130 via the connector system 150 to facilitate circulation of the TTM fluid 112 within the fluid containing layer 420. The fluid containing layer 420 having TTM fluid 112 circulating therein defines a heat sink or a heat source for the patient 50 in accordance with a temperature of the TTM fluid 112.

The pad assembly 120 may include a thermal conduction layer 430 disposed between the fluid containing layer 420 and the patient 50. The thermal conduction layer 430 is configured to facilitate thermal energy transfer between the fluid containing layer 420 and the patient 50. The thermal conduction layer 430 may be attached to the thermal conduction layer 430 along a bottom surface 421 of the fluid containing layer 420. The thermal conduction layer 430 may be conformable to provide for intimate contact with the patient 50. In other words, thermal conduction layer 430 may conform to a contour of the patient 50 to facilitate thermal energy exchange between the thermal conduction layer 430 and the patient 50.

The pad assembly 120 includes an insulation layer 410 disposed on the top side of the fluid containing layer 420. The insulation layer 410 is configured to inhibit thermal energy transfer between the fluid containing layer 420 and the environment. The insulation layer 410 may be attached to the fluid containing layer 420 along a top surface 422 of the fluid containing layer 420. In some embodiments, the insulation layer 410 may comprise one or more openings 411 extending through the insulation layer 410 to provide for coupling of the FDL 130 with the fluid containing layer 420.

The connector system 150 may include an elbow 460 to change the direction of FDL 130 extending away from the connector system 150. As shown, the direction of FDL 130 is shifted from a direction perpendicular to the pad assembly 120 to a direction that is substantially parallel to the pad assembly 120. The elbow 460 also establishes an orientation of a distal portion 461 of the FDL 130 to be substantially parallel to the pad assembly 120 and/or the fluid containing layer 420. The fluid containing layer 420 may comprise one or more internal fluid conduits 426 fluidly coupled to the FDL 130 and the TTM fluid 112 may flow through the internal fluid conduits 426.

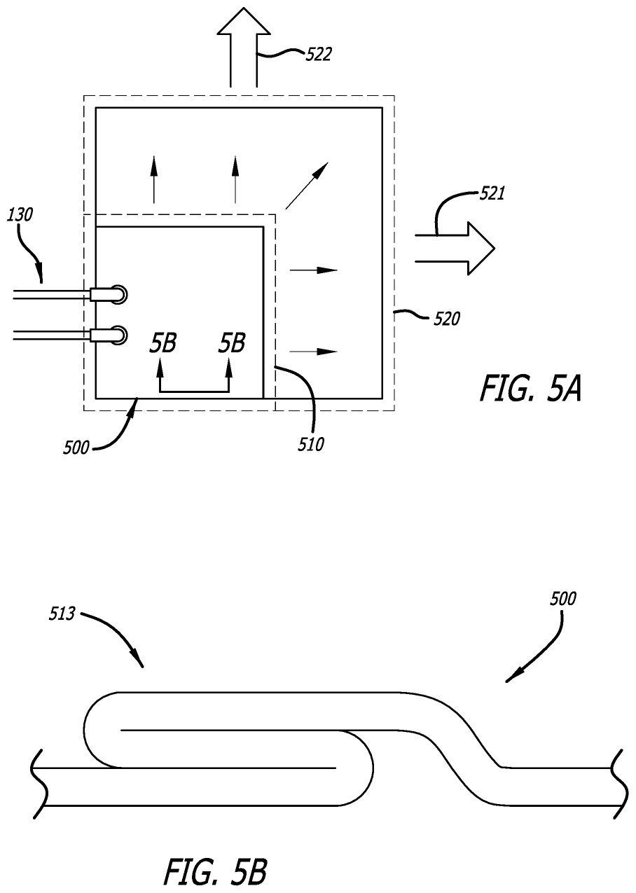

FIGS. 5A and 5B illustrate an expandable pad 500 as may be comprised by the pad assembly 120, in accordance with some embodiments. FIG. 5A is a top view of the expandable pad 500 and FIG. 5B is a cross-sectional side view of a portion of the expandable pad 500 cut along sectioning lines 5B-5B. In the illustrated embodiment, the expandable pad 500 may be converted from a non-expanded state having a first patient contact area 510 to an expanded state having a second patient contact area 520. The second patient contact area 520 may be greater than the first patient contact area 510. The percentage of area increase with respect to the first patient contact area 510 may be up to about 10 percent, 25 percent, 50 percent, 100 percent or more.

In some embodiments, the expandable pad 500 may comprise a generally rectangular two-dimensional shape. In some embodiments, the expandable pad 500 may be expandable along a single dimension such as the length dimension 521 or the width dimension 522. In such an embodiment, the expandable pad 500 may be formed of materials that are generally non-stretchable. As shown in FIG. 5B, the expandable pad 500 may comprise one or more folds 513 defining overlapping portions of the expandable pad 500. As shown, the folds 513 may facilitate expansion expandable pad 500 along the length dimension 521. When in the non-expanded state, the first patient contact area 510 may include one or more overlapping area portions of the expandable pad 500. Expansion of the expandable pad 500 may comprise unfolding portions of the expandable pad 500 so that at least some of the overlapping area portions may be disposed in contact with the patient. In some embodiments, the expandable pad 500 may comprise folds 513 that facilitate expansion expandable pad 500 along the width dimension 522. In some embodiments, the pad 500 may comprise multiple folds 513 oriented in a substantially perpendicular direction with respect to the pad 500 defining a bellows arrangement of the overlapping area portions.

In some embodiments, the expandable pad 500 may be formed of materials that are generally stretchable. Stretchable materials may include elastomeric materials such a silicone, ethylene propylene diene monomer rubber (EPDM), rubber or any other suitably stretchable material. In some embodiments, the expandable pad 500 may be formed of, for example, a viscoelastic material. In some embodiments, the expandable pad 500 may be formed of any of a thermoplastic elastomer based on styrenic block copolymers, a thermoplastic urethane, a thermoplastic ester, a polyether block amide, and/or an olefin block copolymer. When formed of stretchable materials, the expandable pad 500 may be configured to expand along a single dimension (i.e., along the length 521 or the width 522) or along two dimensions such as long the along the length 521 and the width 522.

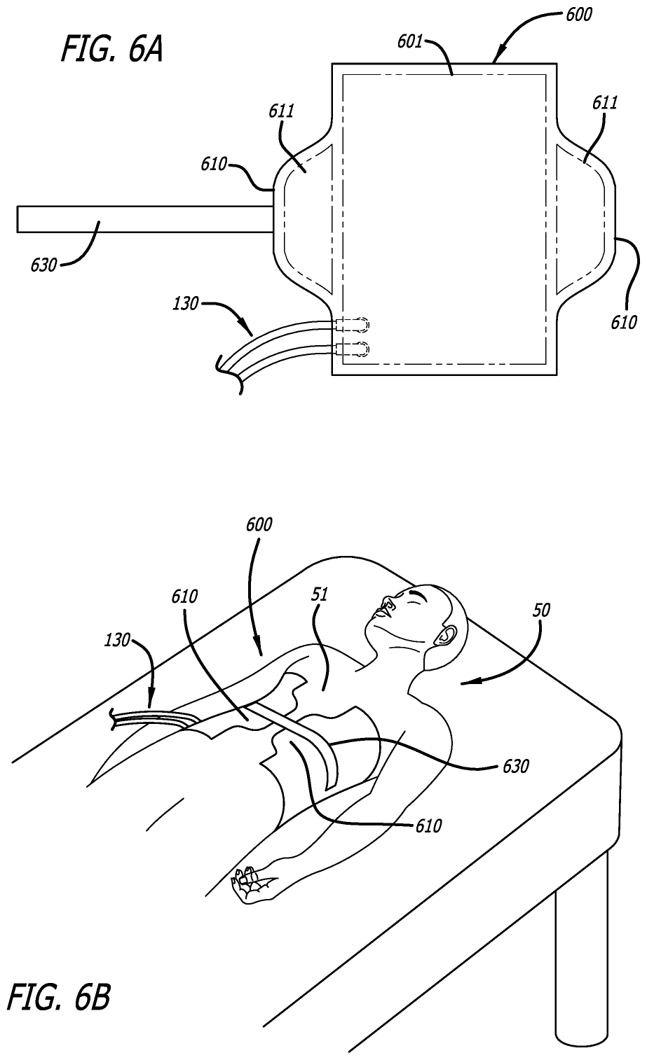

FIGS. 6A and 6B illustrate a thermal pad 600 as may be comprised by the pad assembly 120, in accordance with some embodiments. The thermal pad 600 may be configured to be disposed between the patient 50 and a bed surface. FIG. 6A shows a top view of the pad 600 and FIG. 6B shows the thermal pad 600 as may be applied to the patient 50. In the illustrated embodiment, the thermal pad 600 may be configured to establish a patient contact area 601 with the patient 50 when the patient 50 is laid on the thermal pad 600. In such an instance, a first patient contact area 601 may be defined by the portion of the thermal pad 600 that is disposed directly between the patient 50 and the bed surface.

The thermal pad 600 may comprise lateral extensions 610. The lateral extensions 610 may be configured to extend away from the patient along the bed surface. As such, in some instances, lateral patient contact areas 611 of the lateral extensions may be disposed away from (i.e., not in contact with) the patient 50. The lateral extensions 610 may also be configured to wrap around a portion of a torso 51 of the patient 50 so that the lateral patient contact areas 611 are in contact with the patient 50. In such an instance, a second patient contact area may be defined as the first patient contact are 610 combined with one or both of the lateral patient contact areas 611. In some embodiments, the thermal pad 600 may comprise one or more fastening members 630 (e.g., straps) configured to secure the lateral extensions 610 to the patient 50.

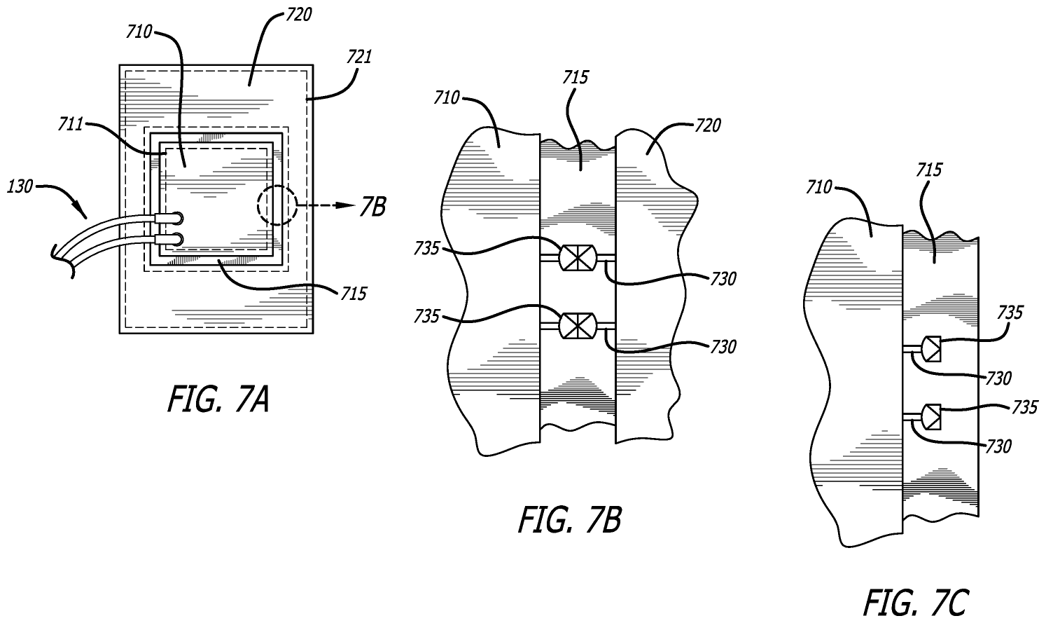

FIGS. 7A-7C illustrate a thermal pad 700 as may be comprised by the pad assembly 120, in accordance with some embodiments. In use, a TTM therapy for a patient may be better facilitated by a thermal pad having a reduced patient contact area. The thermal pad 700 comprises a removable portion 720 coupled to a remaining portion 710. FIG. 7A is top view of the thermal pad 700. The removable portion 720 includes a patient contact area 721 and the remaining portion 710 includes a remaining patient contact area 711. The thermal pad 700 is convertible from a first patient contact area to a second patient contact area, wherein the first patient contact area comprises the removable patient contact area 721 combined with the remaining patient contact area 711, and the second patient contact area comprises only the remaining patient contact area 711.

A separation portion 715 is disposed between the removable portion 720 and the remaining portion 710. The separation portion 715 may comprise materials and/or structure to facilitate separation of the removable portion 720 from the remaining portion 710 by the clinician. In some embodiments, the separation portion 715 may include a perforation (not shown). In other embodiments, the removable portion 720 may be coupled to the remaining portion 710 via 1, 2, 3, 4, or more discreet connecting elements (not shown) extending between the remaining portion 710 and the removable portion 720. In some embodiments, the separation portion 715 may facilitate separation of the removable portion 720 from the remaining portion 710 via the use of a tool by the clinician such as a knife or scissors. In some embodiments, the FDL 130 may be connected to the remaining portion 710.

FIG. 7B is a detail view of a portion of the thermal pad 700 showing one or more fluid conduits 730 extending across the separation portion 715. The fluid conduits 730 may provide for flow of TTM fluid 112 between the removable portion 720 from the remaining portion 710. Each of the fluid conduits 730 may include one or more occlusion mechanisms 735 (e.g., a valves) configured for occlusion of the fluid conduits 730 of the upon removal of the removable portion 720. The occlusion mechanism 735 may be disposed in an open state (i.e., allowing flow through the fluid conduit 730) when the removable portion 720 is coupled to the remaining portion 710, and the occlusion mechanism 735 may be disposed in a closed state (i.e., occluding the fluid conduit 730) when the removable portion 720 is decoupled from the remaining portion 710. FIG. 7C is a detail view of a portion of the pad 700 showing the one or more fluid conduits 730 extending away from the remaining portion 710 after separation of the removable portion 720. The remaining portion 710 may include the occlusion mechanisms 735 to seal the open ends of the fluid conduits 730. In some, the occlusion mechanism 735 may comprise a clamp configure to clamp off the fluid conduit 730. In other embodiments, the occlusion mechanism 735 may comprise a plug configured for insertion within the fluid conduits 730 thereby sealing the open end. Other mechanisms, devices, and methods suitable for occluding the open end of each fluid conduit 730, as may be considered by one of ordinary skill, are included in this disclosure.

As illustrated in FIG. 7A, the removable portion 720 may be coupled to the remaining portion 710 along an entire circumference of the remaining portion 710. In other embodiments, the removable portion 720 may be coupled to the remaining portion 710 along a partial perimeter of the remaining portion 710. In some embodiments, the thermal pad 700 may include more than one removable portion 720 and in such embodiments, one removable portion may be coupled to another removable portion.

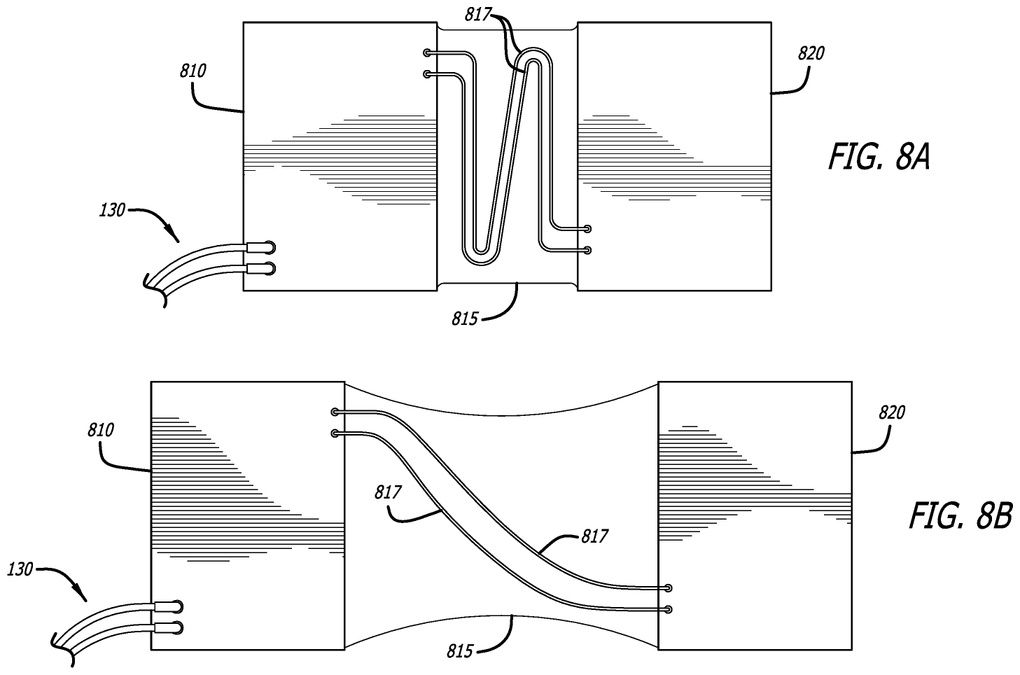

FIGS. 8A and 8B illustrate a thermal pad 800 as may be comprised by the pad assembly 120, in accordance with some embodiments. In some instances, a TTM therapy may be better facilitated by performing thermal energy exchange at separate locations of the patient such as on the front side of the torso and the back side of the torso. The thermal pad 800 comprises a first pad portion 810, a second pad portion, and an extendable member 815 disposed between the first pad portion 810 and the second pad portion 820. FIG. 8A shows the pad 800 in a contracted state and FIG. 8B shows the pad 800 in an extended state.

The extendable member 815 may comprise one or more fluid conduits 817 extending between the first portion 810 and the second portion 820. The fluid conduits 817 may provide for flow of TTM fluid 112 between the first portion 810 and the second portion 820. The extendable member 815 may be formed of a stretchable material such as a stretchable fabric. In other embodiments, the first portion 810, second portion 820, and the extendable member 815 may be formed of the same materials. In some embodiments, the fluid conduits 817 may comprise a serpentine path across the extendable member 815.

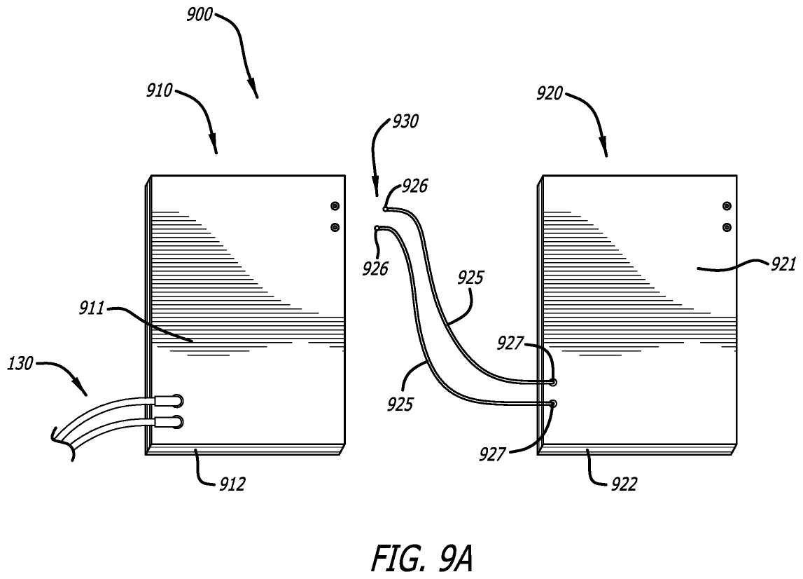

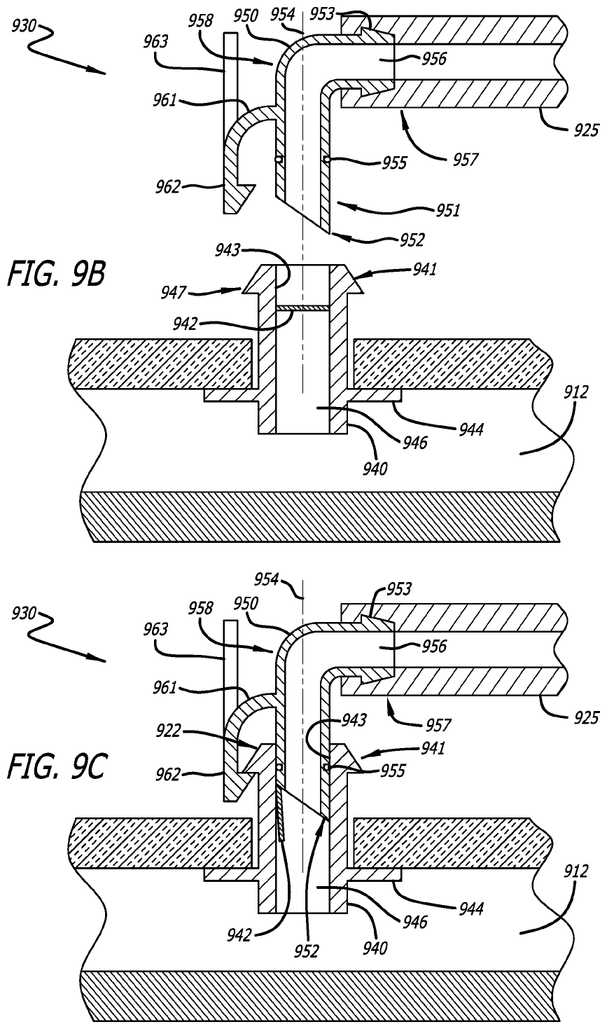

FIG. 9A is an illustration of a thermal pad system 900 as may be comprised by the pad assembly 120, in accordance with some embodiments. In some instances, a TTM therapy may be better facilitated by adding a second thermal pad to a first pad to increase the patient contact area. The thermal pad system 900 include a first pad 910 and a second pad 920 coupleable to the first pad 910 in a daisy chain arrangement. The first pad 910 comprises a first fluid containing layer 912 and a first patient contact area 911, and the second pad 920 comprises a second fluid containing layer 922 and a second patient contact area 921. In use, the clinician may connect to the first pad 910 to increase the patient contract area. As such, the first patient contact area 911 of the thermal pad 900 may be combined with the second patient contact area 921 to define a combined patient contact area.

The thermal pad 900 may comprise one or more fluid conduits 925 extending between the first pad 910 and the second pad 920. The fluid conduits 925 may provide for flow of TTM fluid 112 between the first fluid containing layer 912 of the first pad 910 and the second fluid containing layer 922 of the second pad 920. In use, the clinician may attach and fluidly couple the second pad 920 to the first pad 910. The fluid conduits 925 may be attached and fluidly coupled to the second pad 920 at a second end 927 of the fluid conduits 925. Similarly, the fluid conduits 925 may be attachable and fluidly coupleable to the first pad 910 at a first end 926 of the fluid conduits 925.

FIGS. 9B and 9C illustrate a connector set connector set 930 that may be included with thermal pad system 900 for connecting the second pad 920 to the first pad 910. FIG. 9B shows an exploded cross-sectional side view of the connector set 930 including a pad connector 940 and a complementary fluid line connector 950. FIG. 9C shows an assembled cross-sectional side view of the connector set 930. The fluid line connector 950 is attached to the fluid conduit 925 and the pad connector 640 is attached to a fluid containing layer 912 of the first pad 910. The fluid line connector 950 and the pad connector 940 establish fluid communication between the fluid conduit 925 and the fluid containing layer 912. The connectors 940, 950 are configured to sealably couple together to establish fluid communication between a lumen 956 of the fluid line connector 950 and a lumen 946 of the pad connector 940. The fluid line connector 950 comprises a post portion 951 and the pad connector 940 comprises a post receiving portion 941.

The post portion 951 and the post receiving portion 941 are sized and shaped to form a seal between the pad connector 940 and a fluid line connector 950. The fluid line connector 950 may comprise a sealing member 955 (e.g., an O-ring) to the establish the seal. The pad connector 940 comprises an annular sealing surface 943 to sealably engage the sealing member 955. In some embodiments, the pad connector 940 may comprise the sealing member 955 and the fluid line connector 950 may comprise the annular sealing surface 943. In some embodiments, the post portion 951 may be formed to provide a seal directly with the annular sealing surface 9433. In such embodiments, the sealing member 955 may be omitted.

The post portion 951 and the post receiving portion 941 are sized to provide a sliding fit between the pad connector 940 and a fluid line connector 950. The sliding fit may provide for insertion of the post portion 951 within the post receiving portion 941 and may also provide for rotation of the fluid line connector 950 about the axis 954 with respect to the pad connector 940. In other words, the sliding fit may provide for a rotatable connection between the pad connector 940 and the fluid line connector 950. The fluid line connector 950 may comprise an elbow 958 of about 90 degrees so that a fluid line coupling portion 957 extends in a substantially perpendicular direction away from the axis 654.

In some embodiments, the pad connector 940 may comprise a septum 942 extending across the lumen 946 and forming a seal across the lumen 946. In some embodiments, the post portion 951 may comprise a spike 952 configured to rupture the septum 942 when the fluid line connector 950 is coupled to the pad connector 940 as shown in FIG. 9C. In use the septum 942 may function as a valve, where the valve is closed with the septum 942 is intact and the valve is open when the septum 942 is ruptured. In some embodiments, the septum 942 and the spike 952 may be omitted. In some embodiments, the pad connector 940 may include a valve (not shown) that may be selectively opened and/or closed by the clinician.

The pad connector 940 may comprise a flange 944 to facilitate coupling of the pad connector 940 with the fluid containing layer 912 of the first pad 910. The flange 944 may be coupled to the fluid continuing layer 912 via radio frequency welding, ultrasonic welding, adhesive bonding or any suitable coupling process. The fluid line coupling portion 957 may comprise a barb 953 for coupling the fluid line connector 950 to the fluid conduit 925. Alternatively, the fluid line coupling portion 957 may comprise a bonding socket or any other suitable feature for coupling the fluid line connector 950 to the fluid conduit 925.

The connectors 950, 940 may be configured to couple together via a snap fit. In an exemplary embodiment, the snap fit may comprise one or multiple hooks 962 of the fluid line connector 950 configured to engage with an annular ridge 947 of pad connector 940. The hook 962 may comprise a flexible portion 961 to allow for deflection of the hook 962 upon engagement with the annular ridge 947. The snap fit may be configured to facilitate coupling of the connectors 950, 940 via longitudinal displacement fluid line connector 950 with respect to the pad connector 940. In some embodiments, the snap fit may comprise a release mechanism such as the lever arm 963. Operation of the lever arm 963 may disengage the hook 962 from the annular ridge 962 allowing separation of the fluid line connector 950 from the pad connector 940.

Each of the fluid conduits 922 may comprise a fluid line connector 950 at the first end 926. Similarly, the first pad 910 may comprise one or more corresponding pad connectors 940. In use, the clinician may connect each fluid line connector 950 to the corresponding pad connector 940 to attach and fluidly couple the second pad 920 to the first pad 910. During connection, the clinician may rupture the septum 942 (i.e., open the valve) of each pad connector 940.

In some embodiments, the thermal pad system 900 may include additional thermal pads coupled to each other in the daisy chain arrangement described above. The thermal pad system 900 may include 2, 3, 4, 5, 6, or more thermal pads.

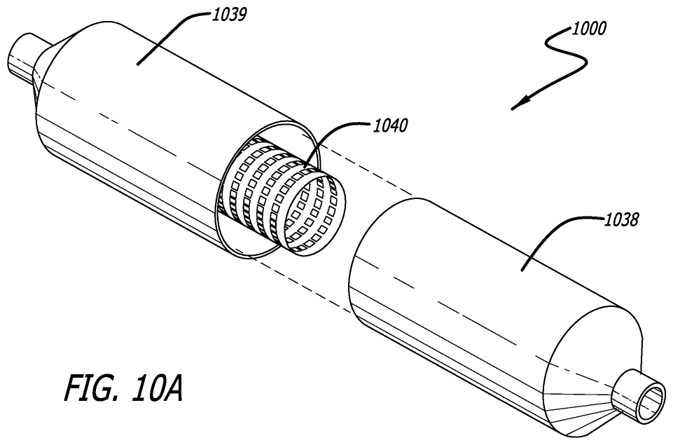

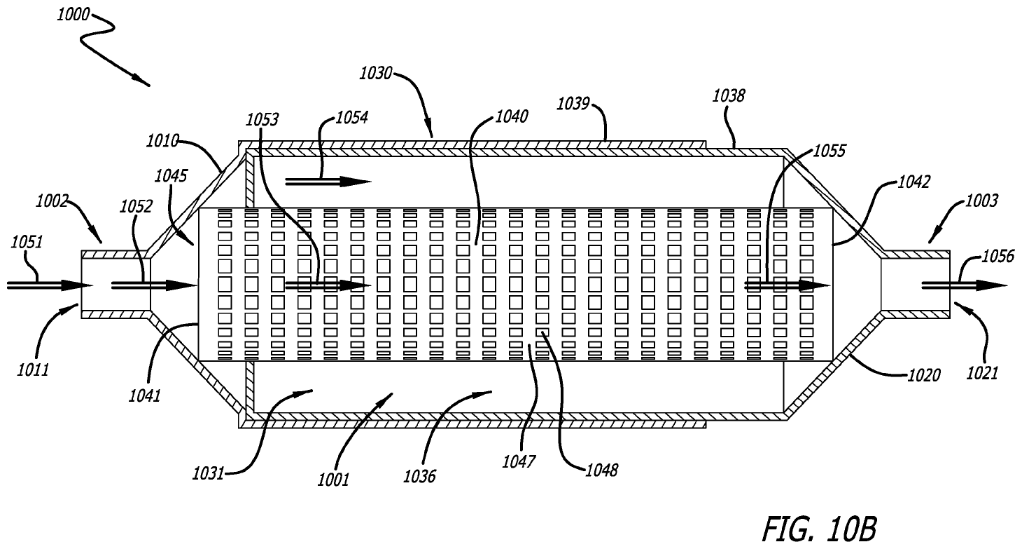

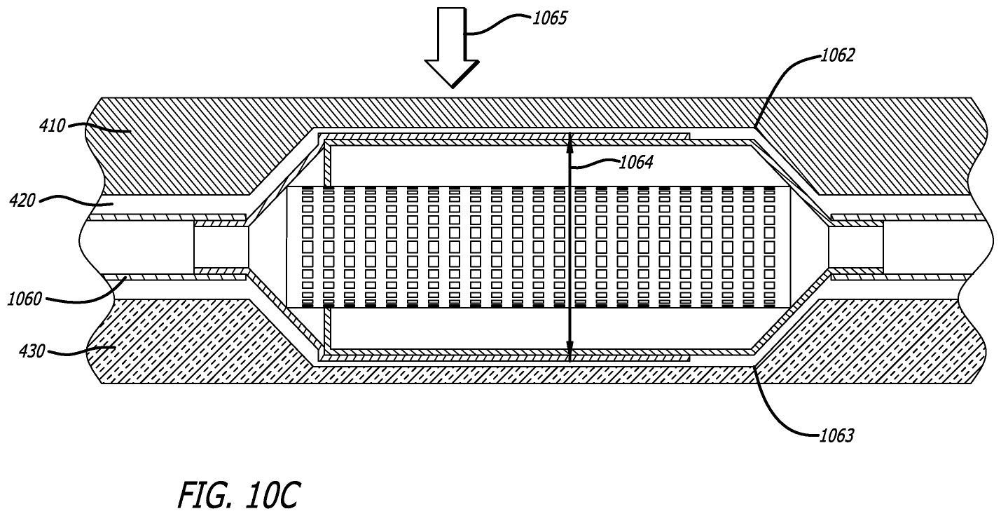

FIGS. 10A and 10B show a filter 1000 that may be included with the TTM system 100. The filter 1000 may be disposed in line with a TTM fluid flow path of the TTM system 100 so that the circulating TTM fluid 112 flows through the filter 1000. The filter 1000 may be configured to remove (i.e., filter out) material/particles having a size of 0.2 microns or larger from the TTM fluid 112 without causing a flow restriction of the TTM fluid 112.

The filter 1000 comprises a longitudinal shape having a flow path 1001 extending from a first end 1002 to a second end 1003. The filter 1000 comprises a diffuser 1010 adjacent the first end 1002, a nozzle adjacent 1020 the second end 1003, and a body 1030 extending between the diffuser 1010 and the nozzle 1020. Along the diffuser 1010, a cross-sectional flow area of the filter 1000 expands from an inlet flow area 1011 to a body flow area 1031 and along the nozzle 1020, the cross-sectional flow area of the filter 1000 contracts from the body flow area 1031 to an outlet flow area 1021. In some embodiments, the inlet flow area 1011 and the outlet flow area 1021 may be substantially equal.

In some embodiments, the body flow area 1031 may be constant along the body 1030. In other embodiments, the body flow area 1031 may vary along a length of the body 1030 such that the body flow area 1031 is greater or less along middle portion of the body 1030 than at the ends of the body 1030. In some embodiments, the body flow area 103 lmay be circular.

The filter 1000 comprises an inner tube 1040 disposed within the body 1030 extending along the length of body 1030. The inner tube 1040 may be coupled to the diffuser 1010 at a first inner tube end 1041 so that TTM fluid 112 entering the filter 1000 at the first end 1002 also enters the inner tube 1040 at the first inner tube end 1041. The inner tube 1040 may be coupled to the nozzle 1020 at a second inner tube end 1042 so that TTM fluid 112 exiting the filter 1000 at the second end 1003 also exits the inner tube 1040 at the second inner tube end 1042.

The inner tube 1040 comprises an inner tube flow area 1045 extending the length of the inner tube 1040. The inner tube flow area 1045 may be greater than the inlet flow area 1011 and/or the outlet flow area 1021. The inner tube flow area 1045 may be constant along the length of the inner tube 1040. In some embodiments, the inner tube flow area 1045 may vary along the length of the inner tube 1040. In some embodiments, the inner tube 1040 may comprise a circular cross section. The inner tube 1040 and the body 1030 may be configured so that the body flow area 1031 comprises a combination of the inner tube flow area 1045 and an annular flow area 1036.

The inner tube 1040 comprises a porous a circumferential wall 1047. The porous wall 1047 may be configured so that TTM fluid 112 may flow through the porous wall 1047, i.e., through the pores 1048 of the porous wall 1047. Consequently, TTM fluid 112 may flow through the porous wall 1047 from the inner tube flow area 1045 to the annular flow area 1036 and from the annular flow area 1036 into the inner tube flow area 1045.

In use, the longitudinal velocity of the TTM fluid 112 may change along the length of the filter 1000. As the volumetric TTM fluid 112 flow through the filter is constant, the longitudinal velocity of the TTM fluid 112 may be at least partially defined by the flow areas of the filter 1000 as described below. The TTM fluid 112 may enter the filter 1000 at a first longitudinal velocity 1051 and decrease along the diffuser so that the TTM fluid 112 enters the inner tube at a second velocity 1052 less than the first longitudinal velocity 1051. At this point, a portion of the TTM fluid 112 may flow through the porous wall 1047 from the inner tube flow area 1045 into the annular flow area 1036 to divide the fluid flow into a third velocity 1053 within the inner tube flow area 1045 and a fourth velocity 1054 within the annular flow area 1036. The fourth velocity 1054 may be less than the third velocity 1053. A portion of the TTM fluid 112 may then flow back into the inner tube flow area 1045 from the annular flow area 1036 to define a fifth velocity 1055 along the inner tube flow area 1045 which may be about equal to the second velocity 1052. The TTM fluid 112 may then proceed along the nozzle 1020 to define a sixth velocity 1056 exiting the filter 1000. In some embodiments, the first velocity 1051 and the sixth velocity 1056 may be about equal.

The filter 1000 may be configured to remove harmful bacteria and viruses from the TTM fluid 112 using sedimentation principles. In use, the filter 1000 may be oriented horizontally so that the direction of fluid flow through the filter 1000 is perpendicular to a gravitational force 1065. In some instances, bacteria, viruses, and other particles within the TTM fluid 112 may have a greater density than the TTM fluid 112 and as such may be urged by the gravitational force 1065 (i.e., sink) in a direction perpendicular to the fluid flow direction. In some instances, particles within the inner tube flow area 1045 may sink toward and through the porous wall 1047 into the annular flow area 1036. Particles within the annular flow area 1036 may then sink toward an inside surface 1031 of the body 1030 and become trapped adjacent the inside surface 1031. The geometry of the filter 1000 may be configured to allow 0.2-micron bacteria/virus particles to fall out of the flow of TTM fluid 112 and become trapped along the inside surface 831.

In some embodiments, the filter 1000 may be configured so that flow of TTM fluid 112 from the inner tube flow area 1045 into the annual flow area 1036 may drag particles through the porous wall 1047. In some embodiments, the inlet flow area 1011, the inner tube flow area 1045, and the annual flow area 1036 may be sized so that the third velocity 1053 is less than about 50 percent, 25 percent, or 10 percent of the first velocity 1051 or less. In some embodiments, the body 1030 and the inner tube 1040 may be configured so that the fourth velocity 1054 is less than the third velocity 1053. In some embodiments, the fourth velocity 1054 may less than about 50 percent, 25 percent, or 10 percent of the third velocity 1053 or less.

In some embodiments, the filter 1000 may be configured so that the flow within the inner tube flow area 1045 is laminar flow, i.e., so that the velocity of the fluid flow adjacent to or in close proximity to an inside surface 1041 of the porous wall 1047 is less than the velocity at a location spaced away from the inside surface 1041. In such an embodiment, the particles may more readily sink toward and through the porous wall 1047.

In some embodiments, the filter 1000 may be configured so that the fluid flow within the annual flow area 1036 is laminar flow, i.e., so that the velocity of the fluid flow adjacent to or in close proximity to inside surface 1031 of the body 1030 is less than the velocity at a location spaced away from the inside surface 1031. In such an embodiment, the particles may more readily sink toward and be trapped along the inside surface 1031.

The filter 1000 may comprise three components including the inner tube 1040 an inner body shell 1038, and an outer body shell 1039. Each of the three components may be formed via the plastic injection molding process. Assembly of the filter 1000 may include capturing the inner tube 1040 within the inner body shell 1038 and the outer body shell 1039 and sliding the inner body shell 1038 into the outer body shell 1039 wherein the fit between the inner body shell 1038 and the outer body shell 1039 is an interference fit.

In some embodiments, the filter 1000 may be disposed within the pad assembly 120. FIG. 10C shows a detail cross-sectional view of the pad assembly 120 including the filter 1000 disposed within the fluid containing layer 420. The filter 1000 is coupled in line with an internal flow path 1060 within the fluid containing layer 420 so that TTM fluid 12 circulating within the pad assembly 120 passes through the filter 1000. The filter 1000 may be sized so that the inlet flow area 1011 and the outlet flow area 1021 are similar to a cross-sectional flow area of the internal flow path 1060 within the fluid containing layer 420.

In some embodiments, a thickness of the fluid containing layer 420 may increase adjacent the filter 1000 to accommodate a body diameter 1064 of the filter 1000. To further accommodate the body diameter 1064, the insulation layer 410 and/or the thermal conduction layer 430 may comprise internal depressions 1062, 1063, respectively.

In some embodiments, one or more filters 1000 may be disposed in line with the flow of TTM fluid 112 at other locations of the TTM system 100. In some embodiments, one or more filters 1000 may be disposed within the TTM module 110. In some embodiments, one or more filters 1000 may be disposed in line with the FDL 130. In some embodiments, the filter 1000 may be disposed in line with a fluid conduit of the pad external to the fluid containing layer 420 such as a conduit extending between the pad connector 652 and the pad assembly 120.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A medical pad for exchanging thermal energy between a targeted temperature management (TTM) fluid and a patient, the medical pad comprising:
a fluid containing layer for containing the TTM fluid, the fluid containing layer configured for circulating the TTM fluid within the containing layer; and
a patient contact surface defining a patient contact area to facilitate thermal energy exchange with the patient, wherein:
the patient contact area is convertible from a first patient contact area to a second patient contact area, and
the second patient contact area is different from the first patient contact area.

2. The medical pad of claim 1, wherein the second patient contact area is at least 10 percent greater than the first patient contact area.

3. The medical pad of claim 2, wherein the patient contact area is expandable from the first patient contact area to the second patient contact area.

4. The medical pad of claim 3, wherein the patient contact area is expandable along a single dimension.

5. The medical pad of claim 3, wherein the medical pad comprises one or more folds to facilitate expansion from the first patient contact area to the second patient contact area patient.

6. The medical pad of any of claim 3, wherein the medical pad comprises a stretchable material to facilitate expansion from the first patient contact area to the second patient contact area.

7. The medical pad of claim 1, wherein the medical pad comprises:
a first pad defining the first patient contact area; and
a second pad connectable to the first pad portion, the second pad including a separate second patient contact area,
wherein when the first patient contact area is converted to the second patient contact area, the second pad is connected to the first pad thereby combining the first patient contact area with the separate second patient contact area to the define the second patient contact area.

8. The medical pad of claim 7, wherein:
the first pad includes a first fluid containing layer,
the second pad includes a second fluid containing layer, and
when the second pad is connected to the first pad the second fluid containing layer is fluidly coupled to the fluid containing layer so that the TTM fluid is circulatable within the second fluid containing layer.

9. The medical pad of claim 8, wherein:
the first pad includes at least one first connector including a valve;
the second pad includes at least one second connector, and
when the second pad is connected to the first pad the at least one first connector is coupled to the at least one second connector the valve of the at least one first connector is opened.

10. The medical pad of claim 1, wherein when the first patient contact area is converted to the second patient contact area a removable portion of the medical pad is separated from a remaining portion of the medical pad.

11. The medical pad of claim 10, wherein the removable portion is disposed along an entire circumference of the remaining portion.

12. The medical pad of claim 10, wherein:
the removable portion comprises a removable fluid containing layer,
the remaining portion comprises a remaining fluid containing layer, and
the medical pad comprises one or more fluid conduits extending between the remaining fluid containing layer and the removable fluid containing layer.

13. The medical pad of claim 12, wherein each of the one or more fluid conduits comprises a valve, and wherein when the first patient contact area is converted to the second patient contact area a removable portion of the one or more conduits is separated from a remaining portion of the one or more conduits, the remaining portion comprising the valve, and the valve is closed.

14. The medical pad of claim 1, wherein the medical pad is configured to be disposed on a bed surface, and wherein the first contact area is defined by a portion of the medical pad disposed between the patient and the bed surface.

15. The medical pad of claim 14, wherein the medical pad comprises one or more lateral extensions extending away from the patient along the bed surface, and wherein when the first contact area is converted to the second contact area at least a portion of the one or more lateral extensions is wrapped around a portion of the patient to define the second contact area.

16. The medical pad of claim 15, wherein the portion of the patient comprises at least a portion of a torso of the patient.

17. The medical pad of claim 15, wherein the medical pad comprises one or more straps to secure the one or more lateral extensions around the portion of the patient.

18. The medical pad of claim 1, wherein a first portion of the medical pad comprising a first portion of the patient contact area is separable from a second portion of the pad comprising a second portion of the patient contact area, and wherein the first portion of the pad is coupled to the second portion of the pad via an extendable member.

19. The medical pad of claim 18, wherein the extendable member comprises one or more fluid conduits extending between the first portion of the pad and the second portion of the pad.

20. The medical pad of claim 1, wherein the fluid containing layer comprises a TTM fluid flow path, and wherein the medical pad further comprises a filter disposed in line with the TTM fluid flow path.

21. A method of providing a targeted temperature management (TTM) therapy to a patient, comprising:
providing a TTM system comprising:
a TTM module configured to provide a TTM fluid;
a thermal pad configured to receive the TTM fluid from the TTM module to facilitate thermal energy transfer between the TTM fluid and a patient; and
a fluid delivery line (FDL) extending between the TTM module and the thermal pad, the FDL configured to provide TTM fluid flow between the TTM module and the thermal pad, wherein:
the thermal pad comprises a patient contact surface defining a patient contact area to facilitate thermal energy exchange with the patient,
the patient contact area is convertible from a first patient contact area to a second patient contact area, and
the second patient contact area is different from the first patient contact area;
applying the pad to the patient; and
delivering TTM fluid from the TTM module to the thermal pad.

22. The method of claim 21, further comprising expanding the pad along a single dimension to convert the first patient contact area to the second patient contact area.

23. The method of claim 21, further comprising stretching a material of the pad to convert the first patient contact area to the second patient contact area.

24. The method of claim 21, further comprising coupling a second thermal pad to the first thermal pad, the first thermal pad defining the first patient contact area, so that the first patient contact area is combined with a separate second patient contact area of the second thermal pad to define the second patient contact area.

25. The method of claim 21, further comprising separating a removable portion of the thermal pad from a remaining portion of the thermal pad to convert the first patient contact area to the second patient contact area.
